(19) United States
(12) Patent Application Publication
(10) Pub. No.: US 2007/0092558 A1
(43) Pub. Date: Apr. 26, 2007

(54) Methods of Preparing Targeted Immunoliposomes

(76) Inventors: George Heavner, Malvern, PA (US); Marian T. Nakada, Malvern, PA (US); Sam Wu, Broomall, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/551,437

(22) Filed: Oct. 20, 2006

Related U.S. Application Data
(60) Provisional application No. 60/728,721, filed on Oct. 20, 2005.

(51) Int. Cl. A61K 31/704; A61K 9/127

(52) U.S. Cl. 424/450; 514/34

(57) ABSTRACT

Methods of preparing targeting ligand bound avidin-lipid vesicles for use in preparing a targeted, therapeutic liposome composition are disclosed. Each vesicle comprises an avidin molecule coupled to the polymer-conjugated biotin which retains multiple free site biotin-binding sites such that the vesicle may be used to further couple a biotinylated-targeting ligand.

### CROSS-REFERENCE TO RELATED APPLICATION

This application claims the benefit of U.S. Provisional Application No. 60/728,721, filed 20 Oct. 2005, the entire contents of which is incorporated herein in its entirety.

### FIELD OF THE INVENTION

The invention relates to methods of preparing a targeted lipid-encapsulated drug delivery system and products prepared by the method. The invention further relates to a method of preparing targeted drug-entrapped liposomal formulations.

### BACKGROUND OF THE INVENTION

Long-circulating liposomes, such as the STEALTH® brand of liposomal technology, have proven suitable delivery systems for targeted drugs to sites of infection, inflammation, and tumors. These PEGylated, sterically-stabilized liposomes show a substantial improvement in their blood circulation half-life in humans over naked liposomal drug particles which are rapidly taken up by the reticulo-endothelial system ((Allen, M. 1991. Biochim. Biophys. Acta 1066 (1), 29-36; Maruyama, K et al. 1992. Biochim. Biophys. Acta 1128 (1), 44-49). Conjugation of a polyethylene glycol (PEG) polymer to the polar head of a phospholipid results in PEGylated liposomes that are more soluble in aqueous environments than their naked counterparts. The hydrophilic PEG molecules surrounding the liposome surface render the structure sterically stable and less immunogenic, protecting it from macrophage uptake and therefore prolonging its circulation time.

Site-specific delivery of drugs can increase therapeutic effects and reduce toxicity. Multiple examples of targeting liposomes with antibodies or antibody fragments, carbohydrates, enzymes and other ligands have been reported. These approaches have advanced site-specific liposome drug carrier technology in applications that include delivery of drug to the brain, lung, tumors or cells of the immune system. Antibody-conjugated liposomes (immunoliposomes) are particularly well suited to this purpose as the targeting is mediated by the high binding affinity of a monoclonal antibody as well as high selectivity for its specific antigen. The overall therapeutic efficacy of targeted liposomes also depends on the ability of the delivery vector to penetrate the target tissue or otherwise reach the desired target cells and deliver the liposomal load. Drug delivery to specific cells by immunoliposomes has proven to be a promising approach for treatment of cancer and other diseases and further exploration of the targeting agents as well as improvements in the manufacturing process for these reagents is warranted.

Immunoliposomes are liposomes conjugated with either whole antibodies, antibody fragments (e.g. Fabs) or re-engineered binding domains (e.g. scFv). It has been observed that using the PEG chains of the sterically-stabilized liposome as a linker between liposome and antibody results in enhanced antibody-antigen binding since steric hindrance is reduced by the PEG shielding the antibody from the lipid layer of the liposome. Methods of attaching PEG polymers to proteins (PEGylating) and other biomolecules are well known in the art.

Methods of attaching antibodies to PEG-coated liposomes through a covalent attachment of the antibody to the free terminus of PEG have been described (Allen. T. M., et al., Biochim. Biophys. Acta, 1237:99-108 (1995); Blume, G., et al., Biochim. Biophys. Acta, 1149:180-184 (1993). In one method, the liposome-PEG-antibody conjugate is included in the lipid composition at the time of liposome formation. This approach has the disadvantage that some of the antibody ligand faces the inner aqueous compartment of the liposome and is unavailable for interaction with the intended target. In another approach, the antibody is PEGylated, the conjugate purified, and subsequently inserted into a preformed liposome. In an alternate procedure, the liposome is preactivated by incorporating, for example PEG-maleide, on its surface. The activated liposome is then contacted by the targeting ligand to be bound, the unreacted head groups must then be quenched, and the resulting mixture purified from unreacted liposomes and unbound protein. In these approaches, a multistep process specific for each targeting ligand must be devised and optimized.

Approaches to developing a universal PEG-modified liposome composition that can be readily attached to a targeting ligand of interest have been undertaken. One proposal for preparing such a universal drug transport vector has been to use the high affinity of biotin-avidin binding as the basis for coupling targeting ligand to liposome. See U.S. Pat. No. 5,171,578 and Schnyder et al. 2004 Biochem J 377: 61-67). However, these methods suffer from certain limitations in either processing procedures or the limitations imposed on the selection of either the lipsomal composition or targeting ligand. Accordingly, a need exists for improved methods for creation of streptavidin-biotin coupled liposomes.

### SUMMARY OF THE INVENTION

One aspect of the invention is a method of preparing an avidin-coupled lipid vesicle comprising: preparing a suspension of biotin-polymer conjugated lipid vesicles; and contacting the biotin-polymer conjugated lipid vesicle suspension with excess avidin or variants thereof to form an avidin-coupled lipid structure displaying biotin-binding sites.

Another aspect of the invention is an avidin-coupled lipid vesicle prepared by the method of the invention, wherein the vesicle displays avidin bound on its surface, the surface-bound avidin being noncovalently attached to said lipid structure and further retaining the ability to bind biotin or biotinylated compounds.

Yet another aspect of the invention is a method of using a ligand-targeted avidin-coupled lipid vesicle prepared by the method of the invention to treat a subject suffering from a condition responsive to the entrapped drug.

### BRIEF DESCRIPTION OF THE DRAWINGS

FIG. 1 is a schematic depicting the method: in step i) amphipathic biotin-polymer-lipids self-associate to form micelles or, alternatively, have been previously incorporated into the lipid layer of a preformed liposome which may further contain entrapped drug; in step ii) the through non-covalent binding, an avidin with multiple biotin binding sites contacts the biotinylated lipid vesicles to form the streptavidin-associated lipid vesicles which retain free biotin binding sites; whereby step iii) biotinylated-targeting ligand may be added to form a targeted-ligand coupled avidin-lipid vesicle.

FIGS. 2A-C are chromatography tracings showing elution profiles of streptavidin-bound lipid separated from biotin-PEG-phospholipid alone and unconjugated streptavidin protein by gel filtration chromatography: elution profile of biotin-PEG(2000)-DSPE (2A), elution profile of streptavidin (2B), and streptavidin conjugated to lipid at a 4:1 molar ratio in the mixture (2C).

FIGS. 3A-B are graphic results of cytotoxicity assays for free doxorubicin and streptavidin-conjugated DOXIL® brand liposomes on MD-MBA 231 human breast tumor cells (A) and A431 human epidermoid cells (B) treated with the same reagents.

FIG. 4 is the elution profiles (refractive index signals) of streptavidin-liposomes formed using 25 μg (lower first peak tracing) and 50 μg (upper first peak tracing) streptavidin and overlaid is the elution peak of 125 μg of biotin-PEG(2000)-DSPE injected alone under the same conditions on SEC (Superose-12/PBS) with calculated MW by static light scattering.

FIGS. 5A-B shows tracings from mass spectrometry analysis of biotinylated murine EGF: murine EGF before biotinylation (A) and biotinylated mEGF (B).

FIGS. 6A-B are histograms used for flow cytometry analysis of biotinylated mEGF captured on streptavidin-liposomes (b-mEGF/SA-lipid complex) bound to MDA-MB 231 tumor cells: detected using goat anti-streptavidin conjugated to FITC (A); or by goat anti-mEGF followed by donkey anti-goat IgG (H+L) conjugated to PE (B).

FIGS. 7A-D are scattergrams from flow cytometry analysis of b-mEGF/SA-lipid complex bound to A431 tumor cells: A431 tumor cells were treated with A) b-mEGF/SA-lipid; B) naked SA-liposomes; C) untreated cells stained with rabbit anti-mEGF and anti-rabbit IgG-APC; and D) untreated cells stained with goat anti-streptavidin-FITC.

### DETAILED DESCRIPTION OF THE INVENTION

All publications, including but not limited to patents and patent applications, cited in this specification are herein incorporated by reference as though fully set forth.

The term “antibodies” as used herein is meant in a broad sense and includes immunoglobulin or antibody molecules including polyclonal antibodies, monoclonal antibodies including murine, human, humanized and chimeric monoclonal antibodies and antibody fragments.

As used herein, “avidin” means a multimeric avidin compound comprising a plurality of very high affinity binding sites for biotin molecules, and includes NeutrAvidin™ brand of biotin binding protein available from Pierce Biotechnology, Inc. (Rockford, Ill.) and streptavidin, a protein produced by Streptomyces avidinii, which has significant conformational and amino acid similarity with avidin, as well as high affinity for biotin. Streptavidin is not glycosylated and reportedly exhibits less non-specific binding to tissues.

“Biotin compound” refers to “biotin” (hexahydro-2-oxo-1H-thieno[3,4-d]imidazoline-4-valeric acid); molecular weight 244 g/mol, also known as a B-complex vitamin, and includes avidin-binding analogs thereof.

“Conjugated” means covalently attached (e.g. via a crosslinking agent. “Coupled” or “bound” means that members of a binding pair are associated, noncovalently, as through a plurality of charged intereactions (ionic bonds) and non-ionic or hydrophobic interactions including VanDerWaals forces such that the bound members retain separate molecular entity.

“Lipid vesicles” refers to any stable micelle or liposome composition comprising vesicle-forming amphipathic lipids including one or two hydrophobic acyl hydrocarbon chains attached to a polar head group and may contain a chemically reactive group, such as an amine, acid, ester, aldehyde or alcohol, at its polar head group.

“Pre-formed liposomes” refers to intact, previously formed unilamellar vesicles (SUVs), large unilamellar vesicles (LUVs) or multi-lamellar vesicles (MLVs) lipid vesicles.

“Therapeutic liposome composition” refers to liposomes which include a therapeutic agent entrapped in the aqueous spaces of the liposomes or in the lipid bilayers of the liposomes.

The present invention relates to a non-covalent (avidin-biotin) coupling procedure combined with a micelle-transfer method for the preparation of streptavidin displaying sterically stabilized small unilamellar vesicles (SUVs), large unilamellar vesicles (LUVs) or multi-lamellar vesicles (MLVs) containing a drug substance. This method provides for the preparation of a targeted-lipid vesicle and, additionally, provides a reagent for the simplified coupling of a plurality of targeting molecules to sterically stabilized liposomes. Targeting ligands such as peptides, Fab fragments, F(ab′)₂, antibodies or enzymes can be attached to this vehicle easily through biotinylation and association with the preformed streptavidin-coupled lipid vesicles which are capable of further biotin binding. Thus, in one embodiment, preformed sterically stabilized liposomes bearing a variety of actives as payload can be targeted as desired in order to affect site-specific therapy.

The avidin-coupled lipid vesicles, in the form of micelles or unilamellar vesicles (SUVs), are mixed with preformed liposomes, and the avidin-coupled lipids are thereby incorporated into the liposomal lipid layers or leaflets. Targeting of avidin-coupled liposome is straight-forward and reproducible. Streptavidin is useful to form the avidin-coupled lipid, since streptavidin has a much lower isoelectric point, pI 5-6, as compared with the basic pI of 10 for avidin. Furthermore, streptavidin is not a glycoprotein, which reduces its potential for nonspecific binding to carbohydrate receptors, such as mannose receptors on cells.

The coupling method and the components of a kit containing the components useful in practicing the method of the invention and products formed by the method of the invention are more fully described hereinbelow.

### Preparation of Targeting Complex

The invention provides a robust method of preparing targeting ligand bound avidin-lipid complexes for use in preparing a targeted, therapeutic liposome composition. Each complex is composed of a (i) a lipid having a polar head group and a hydrophobic tail, (ii) a hydrophilic polymer having a proximal end and a distal end, the polymer attached at its proximal end to the head group of the lipid, (iii) a biotin attached to the distal end of the polymer, (iv) an avidin molecule coupled to the polymer-conjugated biotin and (v) a targeting ligand, which is biotinylated, coupled to the avidin molecule by affinity binding of the biotin group to a free site on said avidin which free site is defined as not bound to said polymer-conjugated biotin.

In one embodiment of the invention, a lipidated-polymer conjugated-biotin, such as, biotinylated PEG(2000)-DSPE, is used as a capture reagent to tether avidin to liposomes. Avidin-coupled lipids have a biotin binding capacity of two to three biotin molecules per avidin. It has been previously demonstrated that biotinylated PEG-DSPE is incorporated into lipsomes (Schnyder, A., et al., Biochem. J. 377:61-67 (2004); Kullberg, E. B., et al., Bioconjugate Chem. 13:737-743 (2002).

The avidin-coupled lipids of the invention are prepared by contacting the avidin molecule with the lipidated-polymer conjugated-biotin with is in the form of a micelle or which has previously been inserted into a preformed therapeutic liposome, in a molar excess of avidin to biotin such that the resulting avidin-coupled micelles retain a plurality of biotin-binding sites on their outer, hydrophilic, surface. In an embodiment of the invention, the molar ratio of avidin to biotin molecules within the micelle or embedded in the liposome is 4:1.

Exemplary lipids useful in the conjugates include distearoyl phosphatidylethanolamine, distearoyl-phosphatidylcholine, monogalactosyl diacylglycerols or digalactosyl diacylglycerols.

The hydrophilic polymer in the conjugates is selected from the group consisting of polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences.

The targeting ligand of the conjugates can be any molecule which has a specificity for a target site which site is a therapeutically relevant membrane, cell, tissue, or organ or a subject and which are further described herein below.

In another embodiment, selecting a targeting conjugate includes determining the ability of the targeting ligand to bind cell surface receptors expressed on the target cell.

In another embodiment, selecting a targeting conjugate is based on (i) the ability of a targeting ligand to bind to cell surface receptors expressed on the target site which is a specific cell type and (ii) the ability of the target cell to internalize liposomes bound to the target cell by binding between the target cell and the targeting ligand.

In another embodiment, a plurality of targeting conjugates each having a unique binding specificity or affinity for a target is selected for use to form a preparation of straptavidin-conjugated liposomes having a plurality of targeting ligand specificities or affinities attached thereto.

Thus, this novel design of streptavidin-conjugated liposomes as a universal drug transport vector provides a versatile platform to refine a liposome-based delivery system.

### Targeting Ligand

A “target” shall mean an in vivo site to which the biotinylated compounds are desired to bind. The actual binding site may be on an organ, tissue, cell or membrane. An exemplary target is a solid tumor (e.g., tumors of the brain or CNS (glioblastomas), lung (small cell and non-small cell), carcinomas of the ovary, prostate, breast and colon as well as other carcinomas and sarcomas) or liquid tumors such as lymphomas (e.g., Non-Hodgkin's lymphoma), leukemias (e.g. acute lymphocytic leukemia) and myelomas (e.g. multiple myeloma, chronic myelogenous leukemia), as well as secondary or metastatic tumors originating from a known or unknown primary tumor, including secondary or metastatic tumors are in those occurring in the lungs, brain, and bone of the host organism. Another exemplary target is a site of infection (e.g. by bacteria, viruses (e.g. HIV, herpes, hepatitis) and pathogenic fungi (Candida sp.) including infectious organisms are Enterobacteriaceae, Enterococcus, Haemophilus influenza, Mycobacterium tuberculosis, Neisseria, gonorrhoeae, Plasmodium falciparum, Pseudomonas aeruginosa, Shigella dysenteriae, Staphylococcus aureus, Streptococcus pneumoniae).

The targeting ligand is a ligand for a binding partner associated with the desired target. In one embodiment, the targeting ligand specifically binds to an extracellular domain of a growth factor receptor. Such receptors are selected from c-erbB-2 protein product of the HER2/neu oncogene, epidermal growth factor receptor, basic fibroblast growth factor receptor, and vascular endothelial growth factor receptor. In another embodiment, the targeting ligand binds a receptor selected from transferrin receptor, a B-cell receptor such as CD19, CD20, CD22, CD37, or CD40; a T-cell receptor such as CD4, an E-selectin receptor; L-selectin receptor; P-selectin receptor; folate receptor; αβ-type integrin receptors such as alphaV-subunit containing integrins; and chemokine receptors such as the CCR2 receptor.

The targeting ligand can be a protein or be a small molecule ligand such as folic acid, pyridoxal phosphate, vitamin B12, sialyl Lewis^{x}, transferrin, epidermal growth factor (EGF) or a fragment thereof, basic fibroblast growth factor, vascular endothelial growth factor (VEGF), VCAM-1, ICAM-1, PECAM-1, an RGD peptide, an NGR peptide, or a chemokine such as CCL2.

TABLE 1
LIGAND-RECEPTOR PAIRS AND
ASSOCIATED TARGET CELL

| LIGAND | RECEPTOR | CELL TYPE |
|---|---|---|
| VEGF | Flk-1, 2 | tumor epithelial cells |
| VCAM-1 | α 4 β 1 integrin | vascular endothelial |
| | | cells |
| Transferrin | Transferrin receptor | endothelial cells (brain) |
| Sialyl- Lewis ^{x} | E, P selectin | activated endothelial |
| | | cells |
| RGD peptides | α ν β 3 integrin | tumor endothelial cells, |
| | | vascular smooth muscle |
| | | cells |
| PECAM-1/CD31 | α ν β 3 integrin | vascular endothelial |
| | | cells |
| Osteopontin | α ν 1 and α ν β 5 | endothelial cells and |
| | integrins | smooth muscle cells in |
| | | atherosclerotic plaques |
| Mac-1 | L selectin | neutrophils, leukocytes |
| Insulin | insulin receptor | pancreatic islet cells |
| ICAM-1 | α L β 2 integrin | vascular endothelial |
| | | cells |
| HIV GP120/41 | Chemokine receptor | macrophages, |
| (Macrophage | CCR5 | dendritic cells |
| tropic isolates) | | |
| HIV GP 120/41 or | CD4 | CD4 + lymphocytes |
| GP120 | | |
| Galactose | Asialoglycoprotein | liver hepatocytes |
| | receptor | |
| Folate | folate receptor | epithelial carcinomas, |
| | | bone marrow stem cells |
| Fibronectin | α ν β 3 integrin | activated platelets |
| EGF | EGF receptor | epithelial cells, |
| | | adenocarcinoma cells |
| basic FGF | FGF receptor | tumor epithelial cells |
| Anti-cell surface | CD19, CD20, CD22, | Activated or malignant |
| receptor antibodies | CD37, | B cells |
| and binding | | |
| fragments thereof | | |
| Anti-cell surface | CD4, CD34, CD40 | Activated or malignant |
| receptor antibodies | | B and T cells |
| and binding | | |
| fragments thereof | | |

An exemplary ligand is an antibody or an antibody fragment including those that bind with high specificity and affinity to an extracellular domain of a growth factor receptor. Exemplary receptors include the c-erbB-2 protein product of the HER2/neu oncogene, epidermal growth factor (EGF) receptor, basic fibroblast growth receptor (basic FGF) receptor and vascular endothelial growth factor receptor, E-, L- and P-selectin receptors, folate receptor, CD4 receptor, CD19 receptor, α/β integrin receptors and chemokine receptors.

In another embodiment, the liposomes may display more than one specificity of targeting ligand. In one aspect of the multiply targeted liposome, the targeting agents are selected based on the desire to direct the lipid-encapsulated drug to multiple binding sites which may be displayed on the same or different cell types, or on cells which may be in more than one stage of growth or differentiation. For example the lipid-encapsulated drug may comprise targeting ligands directed to EGFR (ERBB1) and to Her2 (ERBB2) and thus target both Her2-positive and Her2-negative breast cancer cells.

### Antibodies

An antibody described in this application can include or be derived from any mammal, such as but not limited to, a human, a mouse, a rabbit, a rat, a rodent, a primate, or any combination thereof and includes isolated human, primate, rodent, mammalian, chimeric, humanized and/or CDR-grafted or CDR-adapted antibodies, immunoglobulins, cleavage products and other portions and variants thereof.

Antibodies useful in the embodiments of the invention can be derived in several ways well known in the art. In one aspect, the antibodies can be obtained using any of the hybridoma techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, Antibodies, A Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

The antibodies may also be obtained from selecting from libraries of such domains or components, e.g. a phage library. A phage library can be created by inserting a library of random oligonucleotides or a library of polynucleotides containing sequences of interest, such as from the B-cells of an immunized animal or human (Smith, G. P. 1985. Science 228: 1315-1317). Antibody phage libraries contain heavy (H) and light (L) chain variable region pairs in one phage allowing the expression of single-chain Fv fragments or Fab fragments (Hoogenboom, et al. 2000, Immunol Today 21(8) 371-8). The diversity of a phagemid library can be manipulated to increase and/or alter the immunospecificities of the monoclonal antibodies of the library to produce and subsequently identify additional, desirable, human monoclonal antibodies. For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in a complementarity determining region (CDR) of the variable region of the immunoglobulin polypeptide, and subsequently screened for desirable affinity and neutralization capabilities. Antibody libraries also can be created synthetically by selecting one or more human framework sequences and introducing collections of CDR cassettes derived from human antibody repertoires or through designed variation (Kretzschmar and von Ruden 2000, Current Opinion in Biotechnology, 13:598-602). The positions of diversity are not limited to CDRs but can also include the framework segments of the variable regions or may include other than antibody variable regions, such as peptides.

Other target binding components which may include other than antibody variable regions are ribosome display, yeast display, and bacterial displays. Ribosome display is a method of translating mRNAs into their cognate proteins while keeping the protein attached to the RNA. The nucleic acid coding sequence is recovered by RT-PCR (Mattheakis, L. C. et al. 1994. Proc Natl Acad Sci USA 91, 9022). Yeast display is based on the construction of fusion proteins of the membrane-associated alpha-agglutinin yeast adhesion receptor, aga1 and aga2, a part of the mating type system (Broder, et al. 1997. Nature Biotechnology, 15:553-7). Bacterial display is based fusion of the target to exported bacterial proteins that associate with the cell membrane or cell wall (Chen and Georgiou 2002. Biotechnol Bioeng, 79:496-503).

In comparison to hybridoma technology, phage and other antibody display methods afford the opportunity to manipulate selection against the antigen target in vitro and without the limitation of the possibility of host effects on the antigen or vice versa.

### Biotinylation of Targeting Ligands

Where the targeting ligand is a protein, such as an antibody or fragment thereof, biotin in conveniently conjugated to amine residues present in the protein as epsilon-amino groups of lysine residues or at the amino terminus alpha position by methods known in the art. A variety of coupling or crosslinking agents such as carboiimide, dimaleimide, dithio-bis-nitrobenzoic acid (DTNB), N-succinimidyl-S-acetyl-thioacetate (SATA), and N-succinimidyl-3-(2-pyridyldithio) propionate (SPDP), 6-hydrazinonicotimide (HYNIC), N3 S and N2 S2 can be used in well-known procedures to synthesize biotin amide analogs or biotin compounds. For example, biotin can be conjugated via DTPA using the bicyclic anhydride method of Hnatowich et al, Int. J Appl Radiat Isotop 33:327 (1982). In order to produce predominantly mono-biotinylated targeting ligand, the ratio of biotin to targeting ligand should be small, typically 2:1.

Other compounds useful in attaching biotin to targeting ligands include “biocytin”, a lysine conjugate of biotin, or cadaverine-biotin (N-(5-aminopentyl)biotinamide); biotin ethylenediamine; or activated reagent forms of biotin such as sulfosuccinimidyl 6-(biotinamido) hexanoate (NHS-LC-biotin (which can be purchased from Pierce Chemical Co. Rockford, Ill.) and (6-((biotinoyl)amino)hexanoic acid succinimidyl ester), N-(5-(6-((biotinoyl)amino)hexanoyl) amino) pentylmaleimide); and others available from Biotium, Hayward, Calif.

Another method of preparing a biotinylated targeting ligand is by recombinantly engineering a fusion polypeptide containing the recognition domain for biotin ligase (BirA protein of E. coli, EC 6.3.4.10) which is capable of enzymatic addition of biotin to a specific lysine residue, the MKM motif, within the recognition domain. The recognition domain may be derived from biotinylated proteins derived from a variety of species as it is highly conserved (Cronan Jr., J E. 1990. J Biol Chem 265: 10327-10333; U.S. Pat. No. 4,839,293).

Synthesized biotinylated targeting agents can be characterized using standard methods such as SDS-PAGE, HPLC, MALDI-TOF-MS. Once prepared, candidate biotin derivatives can be screened for ability to bind avidin. In addition, stability can be tested by administering the compound to a subject, obtaining blood samples at various time periods (e.g. 30 min, 1 hour, 24 hours) and analyzing the blood samples for the biotin compound and/or metabolites.

### Lipid Vesicles Containing Therapeutic Agents

Liposomes as well as other micellar lipid vesicles are included in the methods of the invention for incorporation of the targeting ligand in order to act as drug delivery vehicles. The methods of preparation and drug loading procedures for liposomes and the others are well-known in the art. Liposomes can store both nonpolar and polar compounds via interactions with the biocompatible and biodegradable lipid bilayer, or within the aqueous core, respectively.

Lipids suitable for use in the composition of the present invention include those vesicle-forming lipids. Such a vesicle-forming lipid is one which (a) can form spontaneously into unilamellar or bilayer vesicles in water, as exemplified by the diglycerides and phospholipids, or (b) is stably incorporated into lipid structures including unilammellar, bilayered, or rafts.

The vesicle-forming lipids of this type typically have two hydrocarbon chains, usually acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids, cerebrosides and sterols, such as cholesterol.

Cationic lipids are also suitable for use in the liposomes of the invention, where the cationic lipid can be included as a minor component of the lipid composition or as a major or sole component. Such cationic lipids typically have a lipophilic ligand, such as a sterol, an acyl or diacyl chain, and where the lipid has an overall net positive charge. Typicallly, the head group of the lipid carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy) propyl]-N,N,N-trimethylammonium chloride (DOTMA); 3 [N-(N′,N′-dimethylaminoethane)carbamoly]cholesterol (DC-Chol); and dimethyldioctadecylammonium (DDAB).

The cationic vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids. For example, the neutral lipid (DOPE) can be derivatized with polylysine to form a cationic lipid.

In another embodiment, the vesicle-forming lipid is selected to achieve a specified degree of fluidity or rigidity, to control the stability of the liposome in serum, to control the conditions effective for insertion of the targeting conjugate, as will be described, and to control the rate of release of the entrapped agent in the liposome.

Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures.

On the other hand, lipid fluidity is achieved by incorporation of a relatively fluid lipid, typically one having a lipid phase with a relatively low liquid to liquid-crystalline phase transition temperature, e.g., at or below room temperature.

In one embodiment of the method of the invention, the targeted, therapeutic liposome composition of the invention is prepared using pre-formed liposomes and a targeting complex, which are incubated together under conditions effective to achieve insertion of the conjugate into the liposome bilayer. More specifically, the two components are incubated together under conditions which achieve insertion of the conjugate in such a way that the targeting ligand is oriented outwardly from the liposome surface, and therefore available for interaction with its cognate receptor.

Vesicle-forming lipids having phase transition temperatures from approximately 2° C.-80° C. are suitable for use in the pre-formed liposome component of the present composition. By way of example, the lipid distearyl phosphatidylcholine (DSPC) has a phase transition temperature of 62° C. and the lipid hydrogenated soy phosphatidylcholine (HSPC) has a phase transition temperature of 58° C. Phase transition temperatures of many lipids are tabulated in a variety of sources, such as Avanti Polar Lipids catalogue and Lipid Thermotropic Phase Transition Database (LIPIDAT, NIST Standard Reference Database 34).

In one embodiment of the invention, a vesicle-forming lipid having a phase transition temperature between about 30-70° C. is employed. In another embodiment, the lipid used in forming the liposomes is one having a phase transition temperature within a range of 20° C., 10° C. or most typically, 5° C. of the temperature to which the ligand in the targeting ligand avidin-lipid complex can be heated without affecting its binding activity.

It will be appreciated that the conditions effective to achieve insertion of the targeting complex into the liposome are determined based on several variables, including, the desired rate of insertion, where a higher incubation temperature may achieve a faster rate of insertion, the temperature to which the ligand can be safely heated without affecting its activity, and to a lesser degree the phase transition temperature of the lipids and the lipid composition. It will also be appreciated that insertion can be varied by the presence of solvents, such as amphipathic solvents including polyethyleneglycol and ethanol, or detergents.

In an embodiment of the invention, the pre-formed liposomes also include a vesicle-forming lipid derivatized with a hydrophilic polymer. As has been described, for example in U.S. Pat. No. 5,013,556, including such a derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating by presentation of a non-immunogenic outer surface. Such liposomes are also structurally stabilized and are known as sterically-stabilized liposomes

Vesicle-forming lipids suitable for derivatization with a hydrophilic polymer include any of those lipids listed above, and, in particular phospholipids, such as distearoyl phosphatidylethanolamine (DSPE).

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers.

An exemplary hydrophilic polymer chain is polyethyleneglycol (PEG) having a molecular weight between 500-10,000 daltons, more typically between 1,000-5,000 daltons. Methoxy or ethoxy-capped analogues of PEG are also useful hydrophilic polymers, commercially available in a variety of polymer sizes, e.g., 120-20,000 daltons.

Preparation of vesicle-forming lipids derivatized with hydrophilic polymers has been described, for example in U.S. Pat. No. 5,395,619. Preparation of liposomes including such derivatized lipids has also been described, where typically, between 1-20 mole percent of such a derivatized lipid is included in the liposome formulation.

In another embodiment, the liposomes are composed of distearoylphosphatidylcholine (DSPC): cholesterol (52:45 molar ratio), and contain additionally 3 mol % PEG(2000)-DSPE compared to total lipid. The liposomes are prepared by freeze-thaw cycles and extrusion as described (Huwyler, et al. (1996) Proc Natl Acad Sci USA 93: 14164-14169). Essentially, lipids are first dissolved in chloroform or chloroform/methanol 2:1 vol/vol. A lipid film is prepared by vacuum evaporation using a Rotavapor (Büchi, Switzerland). Dried lipid films are hydrated at 40° C. in 0.01 M PBS or 65o in 0.3 M citrate (pH4.0), such that a final lipid concentration of 10 mM is achieved. Lipids are subjected to five freeze-thaw cycles, followed by extrusion (5 times) at 20° C. through a 100 nm pore-size polycarbonate membrane employing an extruder (Avanti Polar Lipids, Alabaster, Ala.). Extrusion is repeated 9 times using a 50 nm polycarbonate membrane. This procedure produces PEG-derived liposomes with mean vesicle diameters of 150 nm. As has been previously demonstrated (Schnyder, et al. (2004) Biochem J 377:61-67), biotinylated loaded liposomes may be prepared by substituting a portion of the PEG-DSPE with linker lipid (biotin-PEG-DSPE) and adding carboxy-fluoroscein at the hydration step.

Insertion of streptavidin-coupled lipid micelles into preformed liposomes is initiated by mixing aliquots of the streptavidin-coupled lipid micelles with preformed liposomes for varying times (1, 2 or 4 hour) and temperatures (37° C., 50° C. or 60° C.). The transfer is performed in a heating block. The procedure for transferring PEG-DSPE micelles into liposomes has been previously reported (Kullberg, E. B., et al., Bioconjugate Chem. 13:737-743 (2002)).

### Therapeutic Agent

A “therapeutic agent” is an agent capable of having a biological effect on a host. Exemplary therapeutic agents are capable of preventing the establishment or growth (systemic or local) of a tumor or infection. Examples include antibiotics, antineoplastic agents, anti-virals, antifungals, toxins (e.g. ricin), radionuclides (e.g. I-131, Y-90, Sm-153), hormone antagonists (e.g. tamoxifen), platinum complexes (e.g. cisplatin), oligonucleotides (e.g. antisense oligonucleotides or silencing (siRNA) olidonucleotides sequences), chemotherapeutic nucleotide and nucleoside analogs (e.g. capecitabine, gemcitabine), boron containing compound (e.g. carborane), photodynamic agents (e.g. rhodamine 123), enediynes (e.g. calicheamicins), and camptothecins (e.g. CPT-11, SN-38, C9), and tyrosine kinase inhibitors (e.g. imatinib mesylate). In an exemplary embodiment for treating or preventing the establishment or growth of a tumor, the therapeutic agent is doxorubicin, a taxane, or cisplatin. In another embodiment for treating or preventing the establishment or growth of a bacterial infection, the therapeutic agent is a quinalone (e.g. levofloxacin), a macrolide (e.g. azithromycin), or a cephalosporin (e.g. cefuroxime) antibiotic. In an exemplary embodiment for treating or preventing the establishment or growth of a viral infection, the therapeutic agent is a reverse transcriptase inhibitor. In an exemplary embodiment for treating or preventing the establishment or growth of a fungal infection, the therapeutic agent is amphotericin B or nystatin.

For the purposes of treating subjects having neoplastic disorders, the entrapped therapeutic agent is, in one embodiment, a cytotoxic drug. Cytotoxic agents are particularly useful as the entrapped agent in liposomes targeted for neoplastic disease indications. The drug may be an anthracycline antibiotic selected from doxorubicin, daunorubicin, epirubicin and idarubicin and analogs thereof. The cytotoxic drug can be a nucleoside analog selected from gemcitabine, capecitabine, and ribavirin. The cytotoxic agent may also be a platinum compound selected from cisplatin, carboplatin, ormaplatin, and oxaliplatin. The cytotoxic agent may be a topoisomerase 1 inhibitor selected from the group consisting of topotecan, irinotecan, SN-38, 9-aminocamptothecin and 9-nitrocamptothecin. The cytotoxic agent may be a vinca alkaloid selected from the group consisting of vincristine, vinblastine, vinleurosine, vinrodisine, vinorelbine and vindesine.

In another embodiment, the entrapped agent is a nucleic acid. The nucleic acid can be an antisense oligonucleotide or ribozyme or a plasmid containing a therapeutic gene which when internalized by the target cells achieves expression of the therapeutic gene to produce a therapeutic gene product.

In another embodiment, the entrapped agent is useful for treating HIV infections and inhibiting HIV replication. The entrapped agent is selected from nucleoside HIV reverse transcriptase inhibitors, non-nucleoside HIV reverse transcriptase inhibitors, HIV protease inhibitors, HIV integrase inhibitors, HIV fusion inhibitors, immune modulators, CCR5 antagonists and antiinfectives is claimed. The nucleoside HIV reverse transcriptase inhibitors may be selected from abacavir, acyclovir, didanosine, emtricitabine, lamivudine, zidovudine, stavudine, atazanavir, and tenofovir. The non-nucleoside HIV reverse transcriptase inhibitors can be efavirenz, nevirapine, and calanolide. The HIV protease inhibitors can be amprenavir, nelfinavir, lopinavir, saquinavir, atazanavir, indinavir, tipranavir, and fosamprenavir calcium. The HIV fusion inhibitors can be enfuvirtide, T-1249, and AMD-3100. The CCR5 antagonists can be TAK-779, SC-351125, SCH-D, UK-427857, PRO-140, and GW-873140.

Anti-HLA-DR coated liposomes containing the HIV protease inhibitor, indinavir, have been disclosed (Gagne et al. (2002) Biochim. Biophys. Acta 1558:198-210).

In one aspect of the present invention are provided immune system modulators which are used as first line therapy or are given in conjunction (prior to, contemporaneously, or following) other types or therapeutic treatments especially for treatment of neoplastic disease and HIV infection or may be immunosuppressant drugs. The immune modulators may be chosen from an interferon (IFN) including an IFNalpha, IFNbeta or IFNgamma-type interferon; granulocyte macrophage colony stimulating factor (GM-CSF), granulocyte colony stimulating sactor (G-CSF), TNFalpha, and IL-2. The immunosuppressant agents of the invention may be chosen from cyclosporine, sirolimus, and mycophenolate mofetil.

### Kits

The biotin-binding avidin-lipid structures of the invention are conveniently used as a component of a kit for preparing targeted lipid vesicles, in particular, targeted sterically-stabilized liposomes. The targeted lipid structure is formed by mixing a biotinylated targeting molecule with the biotin-binding avidin-lipid structure of the invention, such as a liposome. In an embodiment of the invention, the liposome is a sterically stabilized liposome and the targeting agent is an antibody fragment directed to a receptor on the surface of the target cell type.

### Selection Methods

A therapeutic, targeted liposome composition is prepared from the components as follows. A composition specific for a subject suffering from a particular condition, for example a solid tumor of the lung, a bacterial infection or a viral infection, is prepared by selecting a biotinylated targeting ligand from a selection of prepared conjugates. The targeting conjugate is selected either according to knowledge of those of skill in the art of ligand-receptor binding pairs or by obtaining a suitable patient sample, e.g., a fluid sample, a biopsy or the like. The sample is tested by means known to those in the art for expression of a variety of receptors to determine the appropriate targeting ligand.

A pre-formed therapeutic drug entrapped liposome composition is selected based on knowledge of those of skill in the art of the therapeutic agents appropriate for treatment of the particular condition. Alternatively, the therapeutic liposome composition is selected after performing chemosensitivity tests to determine the effect of the entrapped agent on cells of concern obtained from the patient biopsy or fluid sample.

Following selection of the targeting conjugate and of the pre-formed liposome composition, the target-cell sensitized, therapeutic liposome composition for the subject is prepared by combining the two components. As described, the components are combined under conditions effective to achieve affinity binding of the biotin-conjugated targeting ligand to a free site on the avidin-coupled to amphipathic lipidated-polymer conjugated-biotin which is inserted into the liposome bilayer to create the target-cell sensitized liposomes. After coupling is complete, the composition is administered to a patient.

The therapeutic liposomes of the invention may be administered to a patient by intravenous (i.v.) infusion, by subcutaneous (s.c.) injection, by topical application, be taken orally.

The present invention will now be described with reference to the following specific, non-limiting examples.

### EXAMPLE 1

### Preparation of an Avidin-Coupled Micelle

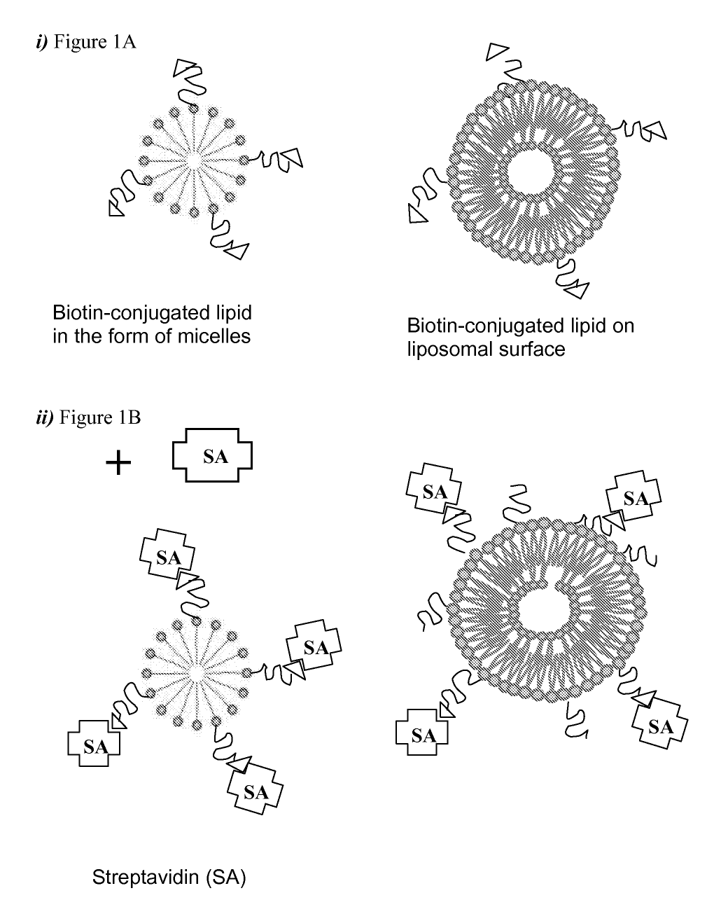

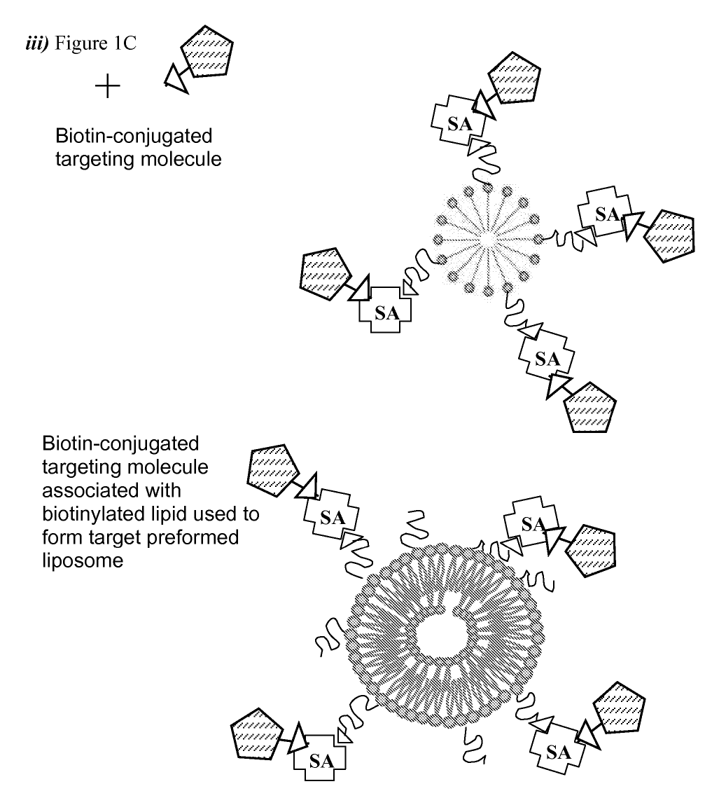

Biotin-PEG(2000)-DSPE, 1,2-Distearoyl-sn-Glycero-3-Phosphoethanolamine-N-[Biotinyl(Polyethylene Glycol)2000] (Avanti Polar Lipids, Inc., Alabaster, Ala.), 5 μmol was dissolved in 1 ml of ethanol/dH₂O (50:50, v/v). Streptavidin (Pierce Biotechnology, Rockford, Ill.) was reconstituted in 20 mM Phosphate buffered saline (PBS), pH 7.2 at a concentration of 1 mg/ml (20 uM). Streptavidin was mixed with Biotin-PEG(2000)-DSPE at a molar ratio of 4:1 (refer to FIG. 1). After incubation for 1 h at 25° C. with gentle shaking, the reaction mixture was purified by GF-250 gel filtration chromatography at a flow rate of 2 ml/min and UV detection at 214 nm. Fractions were collected and analyzed by UV absorbance measurements at 280 nm and SELDI-MS spectrometry.

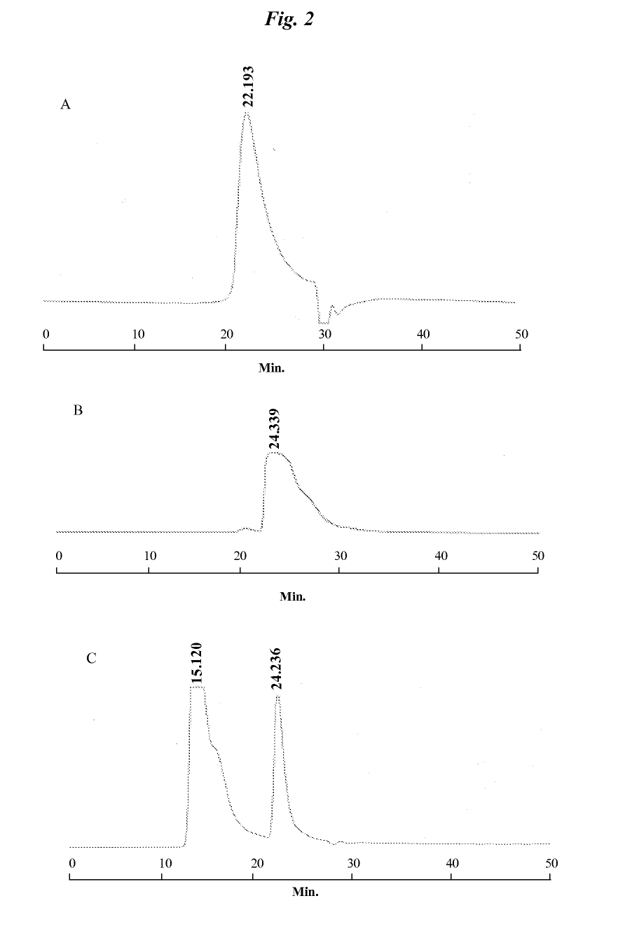

For comparison, three samples; lipid (Biotin-PEG(2000)-DSPE), streptavidin and streptavidin-bound Biotin-PEG(2000)-DSPE, were prepared and analyzed by gel-filtration chromatography using a GF-250 preparative column. The results show that the retention time for biotin-PEG(2000)-DSPE alone is around 22 min (FIG. 2A). The retention time for streptavidin is around 24 min (FIG. 2B). Streptavidin-bound lipid eluted at 15 min (FIG. 2C). These elution profiles revealed that streptavidin-conjugated lipids could be separated from phospholipid alone and unconjugated streptavidin protein by gel filtration chromatography. The resulting fractions collected from the streptavidin-bound lipid were analyzed by measuring the UV absorbance at 280 nm and by SELDI-MS (data not shown).

### EXAMPLE 2

### Formution of a Therapeutic Liposome from an Avidin-Coupled Micelle

DOXIL®, provided by ALZA Corporation (Mountain View, Calif.), is a formulation of doxorubicin encapsulated in polyethylene glycol-coated liposomes (Marina, N. M., et al., Clinical Cancer research, 8: 413-418, (2002)). Insertion of streptavidin-coupled lipid micelles into preformed liposomes was initiated by mixing aliquots of the streptavidin-coupled lipid micelles with Doxil liposomes for 2 hours at 50° C. The total lipid concentration in the reaction was 10 mM. 3 mol % of streptavidin-conjugated lipids compared to total lipids were applied for incorporation to liposomes. The transfer was performed in a heating block. This procedure for transferring PEG-DSPE micelles into liposomes has been previously reported (Kullberg, E. B., et al., Bioconjugate Chem. 13:737-743 (2002)). After the transfer reaction, streptavidin-coupled liposomes were purified by gel filtration on a small column (PD-10) with Sepharose CL-4B gel (Amersham), eluted with 0. 1 M HEPES buffer (pH 7.4). The fractions of streptavidin-coupled liposomes were determined by measuring the UV absorbance at 280 nm for the eluted fractions (0.5 ml each). The concentration of doxorubicin was quantitated by UV measurement at absorbance maximum of 495 nm (ε=12,500) for each fraction of the liposomes entrapping doxorubicin (Banerjee, R., et al., Int. J. Cancer, 112:693-700, (2004)).

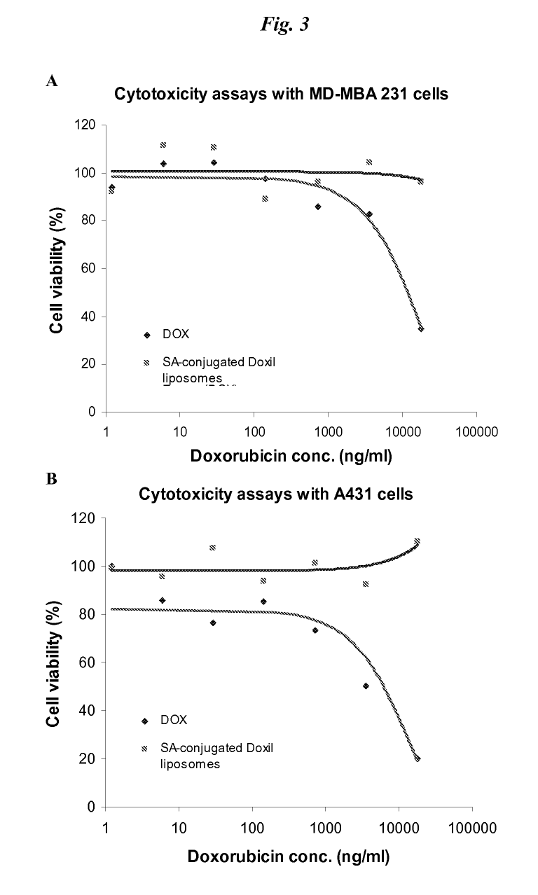

To test the stability of streptavidin-conjugated DOXIL® liposomes, cytotoxicity assays were performed using two tumor cells, MD-MBA 231 (breast cancer cells) and A431 (epidermoid cancer cells). Tumor cells in log growth phase were harvested using 0.05% trypsin-EDTA (Invitrogen, Carlsbad, Calif.). Cell suspension was made at concentration of 1×10⁵ cells/ml. Ten thousand cells in 0.1 ml were added to 96-well plates. After overnight incubation (about 18 hours) for attachment, culture medium was removed and cells were incubated with either free doxorubicin (DOX) or streptavidin-conjugated Doxil liposomes. Both reagents were diluted with growth medium at 1:5 serial concentrations of 18, 3.6, 0.73, 0.144, 0.0288, 0.00576 and 0 μg/ml [DOX]. 0.1 ml for each concentration was added to the cell-attached wells in triplicates. Cells were incubated with test material for 1 hour at 37° C. At the end of incubation, cells were washed three times with 200 μl of growth medium, then, incubated with 100 μl of fresh medium for 72 hours. After 72-hour incubation, the quantity of viable cells was determined using ATPlite™ luminescence assay system (PerkinElmer Life and Analytical Sciences, Shelton, Conn.). Assays were performed according to the manufacture's protocols. Cytotoxicity data showed that there was concentration effects on cell viability with free doxorubicin after 1-hour treatment time, but showed no cytotoxicity effects of streptavidin-conjugated Doxil liposomes treated cells (FIGS. 3A and 3B). These results suggest that there is no leakage of encapsulated doxorubicin from streptavidin-conjugated liposomes after 1-hour incubation with tumor cells. Therefore, the stabilized conjugate was formed.

### EXAMPLE 3

### Characterization of Streptavidin-Coupled Lipid Linker by Static Light Scattering

Biotin-PEG(2000)-DSPE and streptavidin-coupled lipid-linker were characterized by a size exclusion column (SEC) linked to Static Light Scattering (SLS) for solution molecular weight determination. Samples of biotin-PEG(2000)-DSPE loaded at various amounts, ranging from 3 μg to 200 μg, were injected onto a superpose-12 column, pre-equilibrated with PBS, using an Agilent 1100 pump. The eluting peaks were monitored by a UV detector at 280 nm (Agilent); an Optilab-REX refractive index (RI) detector at 690 nm (Wyatt); and a DAWN-EOS light scattering detector (Wyatt). Samples of streptavidin-coupled lipid-linker (bio-PEG-DSPE micells) containing 25 μg and 50 μg of streptavidin were analyzed as described above.

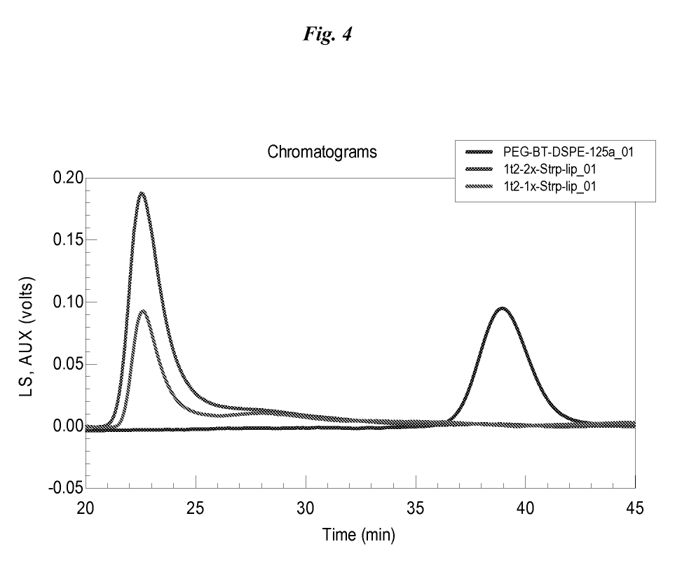

The eluting biotin-PEG(2000)-DSPE peaks were processed by using Astra software (Wyatt), the refractive index signal and a dn/dc value of 0.145 ml/g. The Biotin-PEG(2000)-DSPE has minimal absorbance at 280 nm and was not used for MW determination. All injections of biotin-PEG(2000)-DSPE eluted with a single peak of similar retention time, and a MW of ˜238 kDa. FIG. 4 shows the results of a single injection of biotin-PEG(2000)-DSPE on this column. The retention time for lipid alone is around 39 min and its peak can be detected by refractive index. The MW of 238 kDa calculated from light scattering is consistent with micelle formation composed of ˜79 monomeric units (3016.81 Da per lipid monomer).

Streptavidin-coupled linker-lipid micelles were analyzed by SEC linked to static light scattering using the Astra software along with UV280 nm and RI signals, a dn/dc value of 0.185 ml/g for streptavidin, 0.165 ml/g for the streptavidin-coupled lipid and an extinction coefficient of 1.71 ml/mg,cm for streptavidin. FIG. 3 shows the elution profiles of 25 μg (lower first peak tracing) and 50 μg (upper first peak tracing) peaks from SEC with calculated molecular weights overlaid. Samples predominantly consist of one main peak (r.t.=23 min), however a second smaller peak (r.t.=28 min) is also present. The elution peaks can be detected by both UV280 nm and refractive index. The estimated molecular weights for the first and second peaks appear to be very large. When the 25 μg and 50 μg loads are compared, there is an apparent molecular weights increase with the respective loading amounts, suggesting saturation had not been achieved with 25 ug. Exact molecular weights cannot be assessed without structural verification. These results demonstrated that a streptavidin-associated lipid complex was formed, streptavidin-coupled lipid-linker, and could be separated from the biotin-PEG-DSPE lipid micelles (r.t.=40 min).

### EXAMPLE 4

### Coupled Streptavidin-Lipids

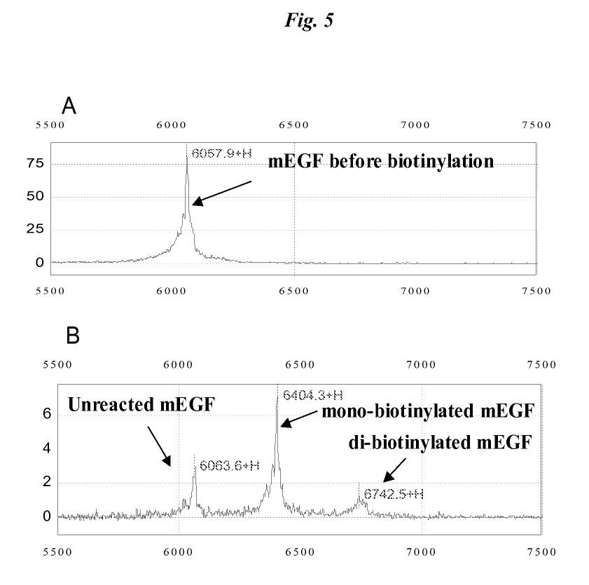

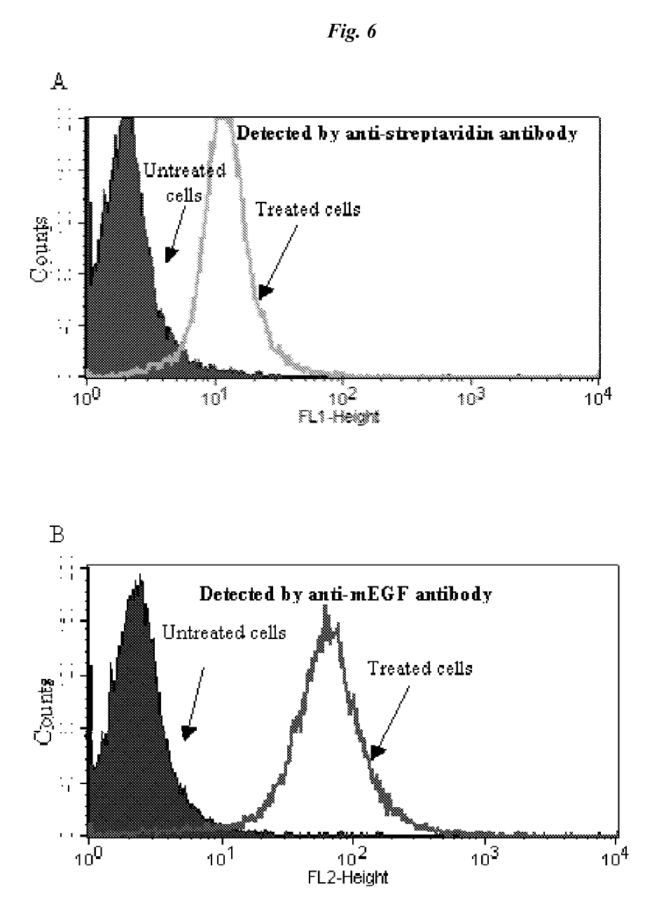

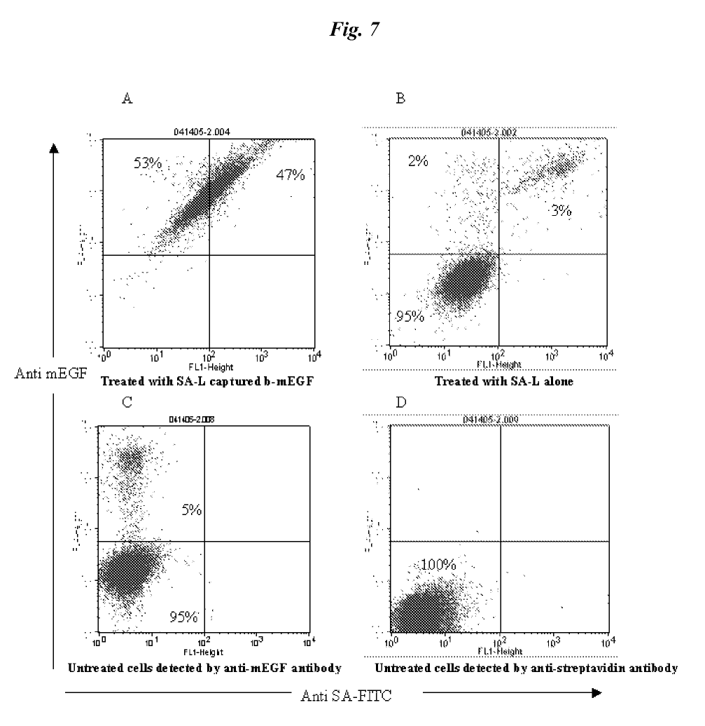

To evaluate whether streptavidin-coupled lipids (SA-lipid) could carry biotinylated ligands capable of specific cell surface receptors binding, biotinylated murine epidermal growth factor (b-mEGF) was selected as a targeting ligand. Murine EGF containing 53 amino acids was purchased from PeproTech (Rocky Hill, N.J.). Dissolved 250 μg EGF in 300 μl PBS buffer, pH 7.4. Immediately before biotinylation, dissolved 2.2 mg of Sulfo-NHS-LC-Biotin (Pierce, Rockford, Ill.) in 400 μl of ultrapure water. To obtain mono-biotinylated mEGF, 2 fold molar excess biotin was incubated with mEGF at room temperature for 30 minutes. 30 μl of 1 M Tris, pH 8.0, was added to the labeling mixture to quench this reaction. The biotinylated mEGF was characterized by mass spectrometry and showed that the final product primarily had mono-biotinylated mEGF, although some unreacted and very little amount of di-biotinylated mEGF were present (FIG. 5A-B). SA-lipid carrying biotinylated murine-EGF peptides were tested using two human tumor cells, MDA-MB 231 (breast cancer cells) and A431 (epidermoid cancer cells), which expressed high level of human EGF receptors on cell surfaces.

Biotinylated mEGF, which is cross-reactive with human EGFR, was incubated with SA-lipid at 1:1 molar ratio for 2 hours at room temperature. The sample mixture or naked SA-lipid were then incubated with MDA-MB 231 or A431 tumor cells for 1 hour at 4° C. In order to assess targeted-lipid particle binding, the tumor cells were then incubated with goat anti-streptavidin conjugated to FITC (Vector Laboratory, Burlingame, Calif.), or goat anti-mEGF Pepro Tech inc., Rocky Hill, N.J.) followed by donkey anti-goat IgG (H+L) conjugated to PE (Jackson ImmunoResearch, West Grove, Pa.) for 45 minutes at 4° C. in the case of MDA-MB 231 cells; or rabbit anti-mEGF (RDL, Flanders, N.J.) followed by donkey anti-rabbit IgG conjugated to APC (Jackson ImmunoResearch, West Grove, Pa.) for 45 minutes at. 4° C. in the case of A431 tumor cells. After incubation, tumor cells were washed 2 times with flow staining buffer (dPBS with 1% BSA, 0.09% sodium Azide). Finally, tumor cells were acquired by FACSCalibur (BD, Franklin Lakes, N.J.) to detect surface-bound streptavidin or mEGF on tumor cells.

FIG. 4 demonstrates that both streptavidin and mEGF peptides were detected by specific antibodies on cell surface. Further, these results indicate that biotinylated mEGF can be captured by SA-lipid and this complex is capable of binding to MDA-MB 231 cell surface presumably through EGF receptor binding. In addition, A431 tumor cells were evaluated. FIG. 5 shows that streptavidin and mEGF peptides were detected on the mEGF/SA-lipid treated cells using specific antibodies (FIG. 5A), but not in the cells treated with naked SA-lipid (FIG. 5B). However, about 3 to 5% of A431 cell population stained positively with rabbit anti-mEGF followed by donkey anti-rabbit IgG-APC (FIGS. 5B and 5C). This observation suggests that A431 cells may express EGF endogenously. In summary, tumor cell binding data demonstrate that SA-lipid can be a ligand delivery vector and target to cell surface receptors.

The present invention now being fully described, it will be apparent to one of ordinary skill in the art that many changes and modifications can be made thereto without departing from the spirit or scope of the appended claims.

1. A method of preparing an avidin-coupled lipid vesicle comprising:
(i) preparing a suspension of biotin-polymer conjugated lipid vesicles; and
(ii) contacting the biotin-polymer conjugated lipid vesicle suspension with excess avidin or variants thereof to form an avidin-coupled lipid vesicle displaying biotin-binding sites.

2. The method of claim 1 further comprising the step of (iii) contacting the avidin-coupled lipid vesicle with a biotinylated targeting ligand.

3. The method of claim 1 wherein the biotin-polymer conjugated lipid vesicle is biotinylated poly(ethylene glycol)-phospholipid.

4. The method of claim 3 wherein the biotinylated poly(ethylene glycol)-phospholipid is biotin-PEG(2000)-distearoylphosphatidylethanolamine (DSPE).

5. The method of claim 1 wherein the biotin-polymer conjugated lipid vesicle suspension is a micellar suspension.

6. The method of claim 1 wherein the biotin-polymer conjugated lipid vesicle suspension is a liposome suspension.

7. The method of claim 1 or 6 wherein the liposomes are drug entrapped liposomes.

8. The method of claim 7 wherein the liposomes are doxorubicin entrapped liposomes.

9. The method of claim 1 wherein the avidin is nonglycosylated avidin.

10. The method of claim 1 wherein the excess of avidin to biotin is 4:1 on a molar basis.

11. An avidin-coupled lipid vesicle prepared by the method of claim 1 wherein the lipid vesicle displays avidin bound on its surface, the surface-bound avidin being noncovalently attached to said lipid vesicle and further retaining the ability to bind biotin or biotinylated compounds.

12. The avidin-coupled lipid vesicle of claim 11 wherein the avidin is noncovalently bound to a conjugated biotin that is integral to the lipid vesicle.

13. The avidin-coupled lipid vesicle of claim 12 comprising biotin-PEG(2000)-(DSPE).

14. The avidin-coupled lipid vesicle of claim 13 wherein the conjugated biotin is integral to the structure of a liposome.

15. The avidin-coupled lipid vesicle of claim 14 wherein the conjugated biotin is integral to the structure of a liposome comprising entrapped drug.

16. The avidin-coupled lipid vesicle of claim 14 or 15 wherein the avidin is further coupled to a biotinylated-targeting ligand.

17. The ligand-targeted avidin-coupled lipid vesicle of claim 16 wherein the biotinylated-targeting ligand binds to EGFR.

18. A kit comprising the prepared lipid vesicle of claim 11 and a biotinylated-targeting ligand for preparing a targeted liposomal vesicle.

19. A method of using the kit of claim 18 to prepare a therapeutic liposome for administration to a subject.

20. A method of using the kit of claim 18 to prepare an experimental composition for the purposes of investigating therapeutic potential of a target liposomal drug.

21. A method of using the kit of claim 18 to prepare a therapeutic liposome for administration to a subject wherein the biotinylated-conjugate is selected based on the results of investigating the presence or absence of receptors on a biopsy specimen from said subject.

22. A method of using the kit of claim 18 to prepare a therapeutic liposome for administration to a subject wherein the biotinylated-conjugate is selected based on the results of investigating the presence or absence of receptors on a biopsy specimen and the therapeutic entrapped agent in said liposome is selected based on the sensitivity of the target site cells to the entrapped agent.

23. A method of using the ligand-targeted avidin-coupled lipid vesicle of claim 11 to treat a subject suffering from a condition responsive to the entrapped drug.
